(19) United States
(12) Patent Application Publication
(10) Pub. No.: US 2013/0274626 A1
(43) Pub. Date: Oct. 17, 2013

(54) MEASURING METHOD FOR SYNCHRONIZING BIO-SIGNALS WITH STIMULATIONS

(71) Applicants: Min-Huims Chiou Chang, Taichung City (TW); Jeng-Ren Duann, Taichung City (TW); Sheng-Chuan Liang, Taichung City (TW); Yung-Jiun Lin, Taichung City (TW); Shih-Che Lo, Taichung City (TW)

(72) Inventors: Min-Huims Chiou Chang, Taichung City (TW); Jeng-Ren Duann, Taichung City (TW); Sheng-Chuan Liang, Taichung City (TW); Yung-Jiun Lin, Taichung City (TW); Shih-Che Lo, Taichung City (TW)

(73) Assignee: UE Technology

(21) Appl. No.: 13/446,188

(22) Filed: Apr. 13, 2012

(51) Int. Cl. A61B 5/00

(52) U.S. Cl. 600/558; 600/559

(57) ABSTRACT

A measuring method for synchronizing bio-signals with stimulations is capable of enhancing utility by using wireless synchronization signal transmission instead of conventional wired synchronization signal transmission and by using a signal processing approach to compensate a delay time in the wireless transmission to obtain a synchronous result. Thereby a stimulus signal corresponds to a biological response signal precisely in order to correctly generate integrated synchronization data of a stimulus organism.

### FIELD OF THE INVENTION

The present invention relates to a measuring method for synchronizing bio-signals with stimulations, and more particularly to a method of synchronous process of measuring bio-signals along with the triggering signals, and/or the onsets of the triggering signals which stimulate and further induce the bio-signals.

### BACKGROUND OF THE INVENTION

Conventional measuring method for a bio-signal in response to a certain stimulus is to input a trigger signal into a stimulus device to produce a stimulus signal, the stimulus signal can be sound or light and so on. The stimulus signal stimulates an analyte organism in different manners. Taking a human body for example, if the stimulus signal is a sound, a tympanum is vibrated; if the stimulus signal is a light, a retina is provoked by light particles. After the human body receives the stimulus signal, a physiological response signal or bio-signal produces. This physiological response signal is generated and then it is measured by a sensing device and stored into a signal processing device. The signal processing device and the stimulus device are connected via some communication channels with or without physical wires, so that the signal processing device can acknowledge the synchronous signal sent from the stimulus device.

Then, the response signal is compared with the synchronization signal of the stimulus signal to realize the induced responses of the analyte organism relative to the forms of stimulus, thus the reaction of the analyte organism can be evaluated based on the induced responses. However, a conventional way of implementing such a mechanism has been mainly based on wired channel and largely limited an arrangement of the signal processing device and the stimulus device and, in term, the free movement of the analyte organism. Besides, if there are analyte organisms and sensing devices, the wire arrangement between them would be very complicated and inconvenient and in turn could limit the applicability of sensing devices.

The present invention has arisen to mitigate and/or obviate the afore-described disadvantages.

### SUMMARY OF THE INVENTION

The primary objective of the present invention is to provide a measuring method for recording bio-signal corresponding to external stimulus, in the same time, wireless transmission mechanism further enhances the utilization of the recording system. However, in order to find the response signal corresponding to the stimulus, a mechanism to synchronize the external stimulus and the recorded bio-signal is necessary.

To achieve the above objective, the present invention first stimulates an analyte organism using a stimulus signal provided from a stimulus device. A synchronization signal is outputted synchronously to a signal processing device at Time 1 via a first wireless transmission interface.

After the analyte organism receives the stimulus signal, a response signal is provoked. The response signal will be measured by a sensing device and then transmitted to a signal processing device.

Right after that, the signal processing device will receive a delayed synchronization signal at Time 2 with known delay time period from Time 1.

The final step is to process the delayed synchronization signal by compensating the known delay time period such that to obtain the correct synchronization signal. The signal processing device then precisely and correctly integrates the synchronization signal with the response signal at Time 1. The result provides an integrated synchronization data of the analyte organism induced by the external stimulation.

Furthermore, the integrated synchronization data (response signal plus synchronization signal) can be transmitted to a signal monitoring processor and stored in a built-in storage device and/or signal acquisition computer through wired or wireless communication channels.

Thereby, the stimulus device can be connected with the signal processing device via the first wireless transmission interface to improve the convenience of usage. Moreover, the signal processing device or the signal monitoring processor compensates the known delay time period of the delayed synchronization signal to obtain the synchronization signal. And then the signal processing device integrates the synchronization signal and the response signal at Time 1 to generate the integrated synchronization data of the analyte organism. This invention fulfills user's requirements.

### BRIEF DESCRIPTION OF THE DRAWINGS

FIG. 1 is a diagram of the first embodiment of the present invention;

FIG. 2 is a diagram of the second embodiment of the present invention.

### DETAILED DESCRIPTION OF THE PREFERRED EMBODIMENTS

A detailed content and a technical description of the present invention are described below. The accompanying drawings are for illustration purpose only. They can not be explained as a limitation of the present invention.

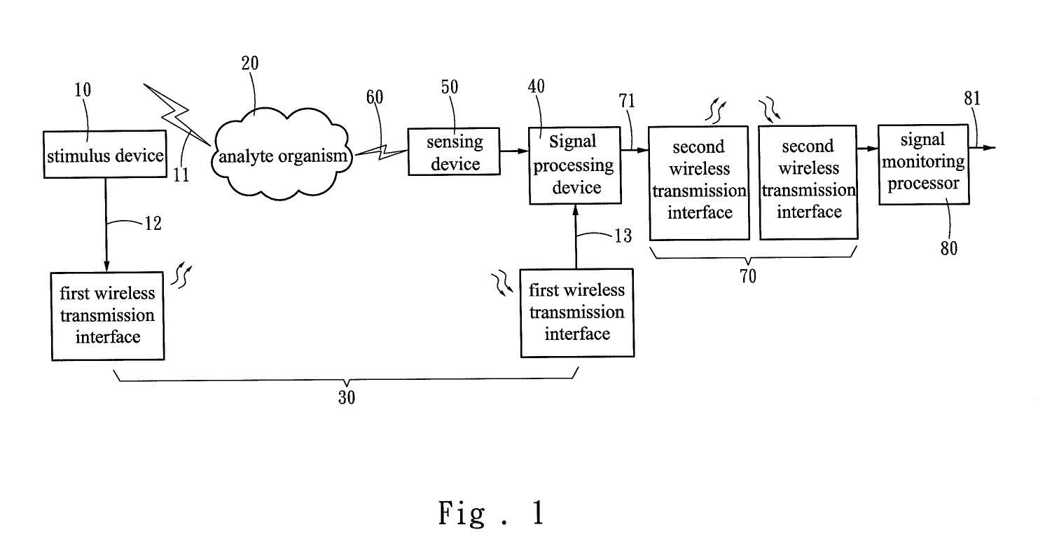

FIG. 1 is a diagram of the first embodiment of the present invention. The present invention first stimulates an analyte organism 20 by a stimulus signal 11 provided from a stimulus device 10 at Time 1. And then a synchronization signal 12 is outputted synchronously to a signal processing device 40 at Time 1 via a first wireless transmission interface 30, wherein the stimulus signal 11 can be a sound or a light that the analyte organism 20 can be stimulated. In order to produce a sound or a light, the stimulus device 10 can be a buzzer or a light source. The stimulus device 10 is driven by a trigger signal such as a sound or a light to generate the stimulus signal 11 and the synchronization signal 12.

The first wireless transmission interface 30 adopts a connection approach selected from the group consisting of WiFi, WiMAX, LTE, UWB, ZigBee, bluetooth, microwave, infrared ray, and radio frequency, and the like, but any other wireless transmission technology are still in the scope of the present invention.

Then, after the analyte organism 20 has received the stimulus signal 11, a response signal 60 is produced in relation to the stimulus signal 11. Then the response signal 60 is sensed via a sensing device 50 in a contact manner or in a non-contact manner, and is transmitted to the signal processing device 40. Thereafter, the signal processing device 40 acquires a delayed synchronization signal 13 at Time 2. Time 2 is a known delay time period from Time 1, which is caused by the first wireless transmission interface 30.

Finally, the signal processing device 40 compensates the known delay time period of the delayed synchronization signal 13 to obtain the correct synchronization signal 12. The signal processing device 40 then integrates the synchronization signal 12 at Time 1 and the response signal 60 to generate the integrated synchronization data 71 of the analyte organism 20.

The present invention also comprises a signal transmitting procedure which allows the integrated synchronization data 71 to be transmitted to a signal monitoring processor 80 via a second wireless transmission interface 70. So that the signal monitoring processor 80 can record, calculate, and analyze the data.

Moreover, the delayed synchronization signal 13 and the response signal 60 can be transmitted to the signal monitoring processor 80 through the signal processing device 40. The signal monitoring processor 80 compensates the delayed synchronization signal 13 to acquire the synchronization signal 12. The synchronization signal 12 at Time 1 and the response signal 60 are integrated to produce integrated synchronization data 81 of the analyte organism 20.

The present invention further comprises a signal processing procedure, which allows the signal processing device 40 to compress and process the integrated synchronization data 71. And then the compressed data is transmitted to the signal monitoring processor 80 via the second wireless transmission interface 70 in order to relieve the load of transmission and to reduce the capacity of signal storage.

Similarly, the second wireless transmission interface 70 adopts a connection approach selected from the group consisting of WiFi, WiMAX, LTE, UWB, ZigBee, bluetooth, microwave, infrared ray, and radio frequency.

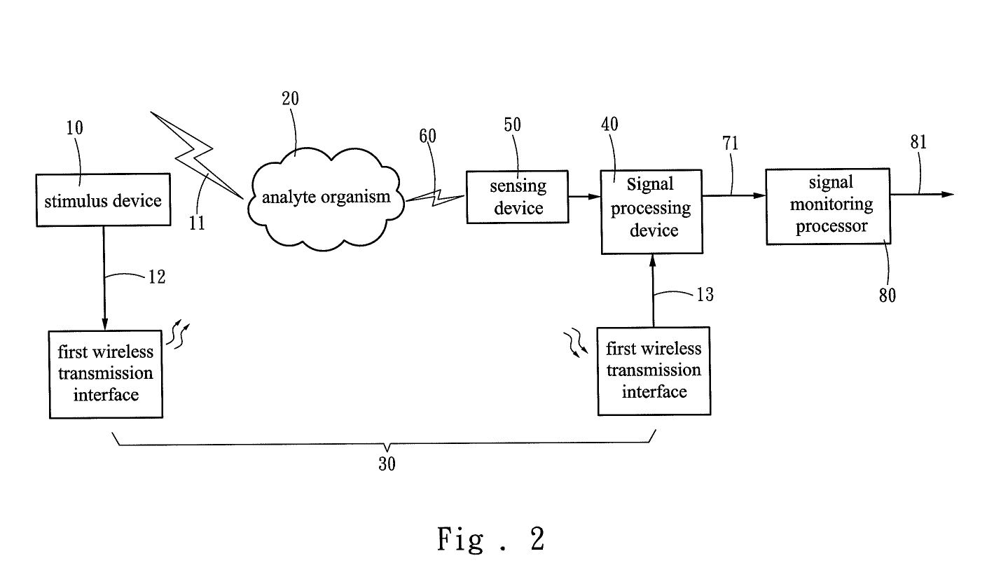

FIG. 2 is a diagram of a second embodiment of the present invention. In this embodiment, in order to transmit the integrated synchronization data 71 outputted from the signal processing device 40 to the signal monitoring processor 80, a wired connection is adopted to allow the signal monitoring processor 80 to obtain a stable signal. Moreover, the signal monitoring processor 80 compensates the delayed synchronization signal 13 to acquire the synchronization signal 12. It also integrates the synchronization signal 12 at Time 1 with the response signal 60 to produce integrated synchronization data 81 of the analyte organism 20.

As above described, the present invention adopts the wireless transmission technology to overcome the inconvenience of wires in most commonly used recording settings and the problem of signal delay in the wireless transmission. So that an accurate integrated synchronization data of an analyte organism can be precisely and correctly generated. This present invention certainly fulfills users' requirements.

Although the foregoing has described certain preferred embodiments, other embodiments will be apparent to those of ordinary skill in the art from the disclosure herein. Additionally, other combinations, omissions, substitutions and modifications will be apparent to the skilled artisan in view of the disclosure herein.

Accordingly, the present invention is not to be limited by the preferred embodiments, but is to be defined by reference to the following claims.

What is claimed is:

1. A measuring method for synchronizing bio-signals with stimulations comprising:
providing a stimulus signal from a stimulus device to an analyte organism at Time 1, and synchronously outputting a synchronization signal to a signal processing device at the Time 1 via a first wireless transmission interface;
generating a response signal from the analyte organism while receiving the stimulus signal, and sensing and transmitting the response signal via a sensing device to the signal processing device;
acquiring a delayed synchronization signal via the signal processing device at Time 2 with known delay time period from the Time 1;
compensating the delayed synchronization signal via the signal processing device to obtain the synchronization signal, and integrating the synchronization signal at the Time 1 and the response signal to generate integrated synchronization data of the analyte organism.

2. The method of claim 1, wherein the first wireless transmission interface adopts a connection approach selected from the group consisting of WiFi, WiMAX, LTE, UWB, ZigBee, bluetooth, microwave, infrared ray, and radio frequency.

3. The method of claim 1, further comprising a signal transmitting procedure which allows the integrated synchronization data to be transmitted to a signal monitoring processor.

4. The method of claim 3, wherein the integrated synchronization data is transmitted to the signal monitoring processor via a second wireless transmission interface.

5. The method of claim 4, wherein the second wireless transmission interface adopts a connection approach selected from the group consisting of WiFi, WiMAX, LTE, UWB, ZigBee, bluetooth, microwave, infrared ray, and radio frequency.

6. The method of claim 3, wherein the integrated synchronization data is wiredly transmitted to the signal monitoring processor.

7. The method of claim 4 further comprising a signal processing procedure which allows the signal processing device to compress and process the integrated synchronization data, and transmit the compressed data to the signal monitoring processor via the second wireless transmission interface.

8. A measuring method for synchronizing bio-signals with stimulations comprising:
providing a stimulus signal from a stimulus device to an analyte organism at Time 1, and synchronously outputting a synchronization signal to a signal processing device at the Time 1 via a first wireless transmission interface;
generating a response signal from the analyte organism while receiving the stimulus signal, and sensing and transmitting the response signal via a sensing device to the signal processing device;
acquiring a delayed synchronization signal via the signal processing device at Time 2 with known delay time period from the Time 1;
receiving the delayed synchronization signal and the response signal via the signal processing device, and transmitting the delayed synchronization signal and the response signal to a signal monitoring processor; and
compensating the delayed synchronization signal via the signal monitoring processor to obtain the synchronization signal, and integrating the synchronization signal at the Time 1 and the response signal to generate integrated synchronization data of the analyte organism.

9. The method of claim 8, wherein the first wireless transmission interface adopts a connection approach selected from the group consisting of WiFi, WiMAX, LTE, UWB, ZigBee, bluetooth, microwave, infrared ray, and radio frequency.

10. The method of claim 8, wherein after the delayed synchronization signal and the response signal are received by the signal processing device, they are transmitted to the signal monitoring processor through a second wireless transmission interface.

11. The method of claim 10, wherein the second wireless transmission interface adopts a connection approach selected from the group consisting of WiFi, WiMAX, LTE, UWB, ZigBee, bluetooth, microwave, infrared ray, and radio frequency.

12. The method of claim 10 further comprising a signal processing procedure which allows the signal processing device to compress and process the integrated synchronization data, and transmit the compressed data to the signal monitoring processor via the second wireless transmission interface.

13. The method of claim 8, wherein after the delayed synchronization signal and the response signal are received by the signal processing device, they are wiredly transmitted to the signal monitoring processor.
